(19) United States
(12) Patent Application Publication
(10) Pub. No.: US 2010/0008876 A1
(43) Pub. Date: Jan. 14, 2010

(54) Artificial nail composition having improved curability

(76) Inventors: Hisaki Tanaka, Kyoto-shi (JP); Mikito Deguchi, Kyoto-shi (JP)

Correspondence Address: WENDEROTH, LIND & PONACK, L.L.P., 1030 15th Street, N.W.,, Suite 400 East, Washington, DC, 20005-1503, US

(21) Appl. No.: 12/458,389

(22) Filed: Jul. 10, 2009

(30) Foreign Application Priority Data
Jul. 10, 2008 (JP) 2008-180270
Jun. 26, 2009 (JP) 2009-152449

(51) Int. Cl. A61K 8/55; A61Q 3/00

(52) U.S. Cl. 424/61

(57) ABSTRACT

An object of the present invention is to provide an artificial nail composition having improved curability. Another object of the present invention is to provide an artificial nail composition which causes less yellowing and has excellent internal curability.

The artificial nail composition comprises:
a component (a): a compound having at least one radical polymerizable unsaturated double bond in the molecule, and a component (b): a photopolymerization initiator selected from (i) an acylphosphine oxide-type photopolymerization initiator and (ii) a mixture of an acylphosphine oxide-type photopolymerization initiator and an α-hydroxyalkylphenone-type photopolymerization initiator, wherein the amount of the component (b) is 0.05 to 4.00 parts by weight with respect to 100 parts by weight of the component (a). It is preferred that the acylphosphine oxide-type photopolymerization initiator is monoacylphosphine oxide and/or bisacylphosphine oxide, and the α-hydroxyalkylphenone type polymerization initiator is 1-hydroxy-cyclohexyl-phenyl-ketone or 2-hydroxy-2-methyl-1-phenyl-propan-1-one.

### BACKGROUND OF THE INVENTION

1. Field of the Invention

The present invention relates to an artificial nail composition having improved curability.

2. Description of the Related Art

Nail art means makeup or decoration of nails of the hands and feet. A shop for nail art is called a nail salon and a nail art technician is called a nail technician. Various nail art goods are commercially available and there are many women who perform nail art with skill equivalent to a professional.

In the 19th century, a lacquer coating material for automobiles was invented in the United States and a currently used manicure was developed by applying this technique.

A manicure utilizing the lacquer coating material is now widely spread, but has a problem that it has poor adhesion to a natural nail and may be peeled or removed within a short period after coating. Therefore, an artificial nail material utilizing a dental resin which is polymerizable at a normal temperature has been developed.

An artificial nail material utilizing a dental resin which is polymerizable at a normal temperature has excellent strength and durability when compared with a manicure utilizing a lacquer coating material and is therefore accepted by some professional nail technicians. However, due to irritation caused by an acryl monomer, irritating odor and poor operability, such an artificial nail material has not been sufficiently spread among general nail technicians.

Recently, a gel nail obtained by improving odor irritation and operability of an artificial nail utilizing a dental resin which is polymerizable at a normal temperature is popular in the market. A gel nail, which is now commercially available, is a high-viscosity liquid material containing a (meth)acryl-based monomer and a photopolymerization initiator as main components and is cured by irradiation with ultraviolet rays. These commercially available gel nails cause less odor irritation or skin irritation and also have more excellent operability when compared with the above-described artificial nail material utilizing a dental resin which is polymerizable at a normal temperature. As a result, they are accepted by the majority of general nail technicians. However, commercially available gel nails are ultraviolet curable systems and there is a fear of an adverse influence of ultraviolet rays on the human body. Furthermore, yellowing of a cured body occurs and thus it is impossible to meet aesthetic demands of consumers.

International Publication No. WO 00/076366 discloses a method of coating a photocurable resin composition to form an uncured layer on a nail and curing the layer by irradiating with visible rays. Although improvement with respect to use of ultraviolet rays harmful to the human body is recognized, improvement with respect to curability, yellowing, internal curing, etc. of the composition are not recognized.

JP-A No. 11-21212 discloses an artificial nail composition comprising a monomer containing a vinyl monomer and a polymerization initiator, in which a polymerization initiator which is decomposed by absorbing visible rays is used as the polymerization initiator. Although improvement with respect to use of ultraviolet rays harmful to the human body is recognized, improvement with respect to curability, yellowing, internal curing, etc. of the composition are not recognized.

### SUMMARY OF THE INVENTION

An object of the present invention is to provide an artificial nail composition having improved curability. Another object of the present invention is to provide an artificial nail composition which causes less yellowing and has excellent internal curability.

The present invention provides:

(1) An artificial nail composition comprising:

a component (a): a compound having at least one radical polymerizable unsaturated double bond in the molecule, and

a component (b): a photopolymerization initiator selected from (i) an acylphosphine oxide-type photopolymerization initiator and (ii) a mixture of an acylphosphine oxide-type photopolymerization initiator and an α-hydroxyalkylphenone-type photopolymerization initiator, wherein the amount of the component (b) is 0.05 to 4.00 parts by weight with respect to 100 parts by weight of the component (a).

The present invention also provides:

(2) The artificial nail composition according to (1), wherein the component (b) the acylphosphine oxide type photopolymerization initiator is monoacylphosphine oxide and/or bisacylphosphine oxide and the α-hydroxyalkylphenone-type polymerization initiator is 1-hydroxy-cyclohexyl-phenyl-ketone or 2-hydroxy-2-methyl-1-phenyl-propan-1-one.

The present invention provides:

(3) The artificial nail composition according to (1) or (2), wherein a visible or ultraviolet light source is used as a photopolymerization light source upon curing.

The artificial nail composition of the present invention enables photopolymerization with visible rays which are harmless to the human body, in addition to conventional ultraviolet rays. Furthermore, since a cured body causes less yellowing, it becomes possible to meet aesthetic demands of consumers. Since it is effective for internal curing of a composition containing a pigment, the artificial nail composition of the present invention can easily impart special beautiful external appearance and a function required as an artificial nail to fingertips. Furthermore, excellent surface gloss can be obtained by good surface curability.

### DETAILED DESCRIPTION OF THE INVENTION

A compound having at least one radical polymerizable unsaturated double bond in the molecule (a) in the present invention can be used by selecting from known monofunctional and polyfunctional polymerizable monomers. Typical examples of the compound, which is preferably used, include polymerizable monomers having an acryloyl group and/or a methacryloyl group or urethane(meth)acrylates. In the present invention, both an acryloyl group-containing polymerizable monomer and a methacryloyl group-containing polymerizable monomer are comprehensively represented by (meth)acrylate or (meth)acryloyl.

Specific examples thereof include: monofunctional monomers such as methoxyethylene glycol(meth)acrylate, methoxypolyethylene glycol(meth)acrylate, phenoxyethylene glycol (meth)acrylate, phenoxypolyethylene glycol(meth)acrylate, 2-hydroxy-3-phenoxypropyl (meth)acrylate, 2-(meth)acryloyloxyethylsuccinic acid, 2-(meth)acryloyloxyethylphthalic acid, 2-(meth)acryloyloxypropylhexahydrophthalic acid, stearyl(meth)acrylate and 3-chloro-2-hydroxypropyl(meth)acrylate; difunctional monomers such as 1,6-hexanediol di(meth)acrylate, 1,9-nonanediol di(meth)acrylate, 1,10-decanediol di(meth)acrylate, neopentyl glycol di(meth)acrylate, 2-methyl-1,8-octanediol di(meth)acrylate, glycerin di(meth)acrylate, ethylene glycol di(meth)acrylate, polyethylene glycol di(meth)acrylate, propylene glycol di(meth)acrylate, polypropylene glycol di(meth)acrylate, ethoxylated polypropylene glycol di(meth)acrylate, ethoxylated propylene glycol di(meth)acrylate, ethoxylated bisphenol A di(meth)acrylate, propoxylated bisphenol A di(meth)acrylate, propoxylated ethoxylated bisphenol A di(meth)acrylate and tricyclodecanedimethanol di(meth)acrylate; and

tri- or higher polyfunctional monomers such as trimethylolpropane tri(meth)acrylate, ethoxylated trimethylolpropane tri(meth)acrylate, ethoxylated glycerin tri(meth)acrylate, ditrimethylolpropane tetra(meth)acrylate, pentaerythritol tri(meth)acrylate, pentaerythritol tetra(meth)acrylate, propoxylated pentaerythritol tetra(meth)acrylate, ethoxylated pentaerythritol (meth)acrylate, dipentaerythritol hexa(meth)acrylate and ethoxylated isocyanuric acid tri(meth)acrylate.

Urethane(meth)acrylates used in the present invention have at least two or more of acryloyl groups and/or methacryloyl groups and an urethane group in one molecule. For example, 7,7,9-trimethyl-4,13-dioxo-3,14-dioxo-5,12-diaza-hexadecane-1,16-diol-dimethacrylate (hereinafter, referred to as “UDM”), 1,6-bis[(2-phenoxy-2′-acryloxy)isopropyloxy)carbonylamino]hexane (hereinafter, referred to as “UDMA”), 1,1,1-tri[6-{1-acryloxy-3-phenoxy)isopropyloxycarbanylamino}hexylcarbamoyloxymethyl]propane (hereinafter, referred to as “URO”) are included.

In addition to the above-described (meth)acrylate-based polymerizable monomers, other polymerizable monomers, oligomers or polymers of monomers, for example, monomers having at least one polymerizable group in the molecule may be used without any limitation depending on the purposes of the artificial nail composition. These monomers may have a substituent such as an acidic group or a fluoro group in the same molecule.

In the present invention, a compound having at least one radical polymerizable unsaturated double bond in the molecule (a) includes not only a single component, but also a mixture of a plurality of polymerizable monomers. When the polymerizable monomer has a very high viscosity at room temperature or is a solid, it is preferred to use as a mixture of a low-viscosity polymerizable monomer and the polymerizable monomer. Not only two or more kinds, but also three or more kinds may be used in combination.

Further, it is possible to include an acidic component having at least one radical polymerizable unsaturated double bond in the molecule as (a) the compound having at least one radical polymerizable unsaturated double bond in the molecule. The component is preferably an acidic phosphorus compound having at least one radical polymerizable unsaturated double bond in the molecule. The compound having a P—OH bond has a phosphoric acid monoester group, a phosphoric acid diester group, a phosphonic acid group, a phosphonic acid monoester group, a phosphorous acid monoester group, a phosphinic acid group or a pyrophosphoric acid group, and a compound having a phosphoric acid monoester group, a phosphoric acid diester group or a phosphonic acid group is particularly preferred.

The acylphosphine oxide-type photopolymerization initiator (b) in the present invention includes a polymerization initiator having one acyl group and a polymerization initiator having two acyl groups, which are respectively referred to as monoacylphosphine oxide and bisacylphosphine oxide and both of them can be employed.

Examples thereof include acylphosphine oxides such as 2,4,6-trimethylbenzoyldiphenylphosphine oxide, 2,6-dimethoxybenzoyldiphenylphosphine oxide, 2,6-dichlorobenzoyldiphenylphosphine oxide, 2,4,6-trimethylbenzoylphenylphosphinic acid methyl ester, 2-methylbenzoyldiphenylphosphine oxide, pivaloylphenylphosphinic acid isopropyl ester, bis-(2,6-dichlorobenzoyl)phenylphosphine oxide, bis-(2,6-dichlorobenzoyl)-2,5-dimethylphenylphosphine oxide, bis-(2,6-dichlorobenzoyl)-4-propylphenylphosphine oxide, bis-(2,6-dichlorobenzoyl)-1-naphthylphosphine oxide, bis-(2,6-dimethoxybenzoyl)phenylphosphine oxide, bis-(2,6-dimethoxybenzoyl)-2,4,4-trimethylpentylphosphine oxide, bis-(2,6-dimethoxybenzoyl)-2,5-dimethylphenylphosphine oxide, bis-(2,4,6-trimethylbenzoyl)phenylphosphine oxide and bis-(2,5,6-trimethylbenzoyl)-2,4,4-trimethylpentylphosphine oxide. Among these, 2,4,6-trimethylbenzoyldiphenylphosphine oxide and bis(2,4,6-trimethylbenzoyl)phenylphosphine oxide are preferred.

The α-hydroxyalkylphenone-type photopolymerization initiator (b) in the present invention is preferably 1-hydroxy-cyclohexyl-phenyl-ketone, 2-hydroxy-2-methyl-1-phenyl-propan-1-one, 1-[4-(2-hydroxyethoxy)-phenyl]-2-hydroxy-2-methyl-1-propan-1-one or 2-hydroxy-1-{4-[4-(2-hydroxy-2-methyl-propionyl)-benzyl]phenyl}-2-methyl-propan-1-one, and more preferably 1-hydroxy-cyclohexyl-phenyl-ketone or 2-hydroxy-2-methyl-1-phenyl-propan-1-one.

Examples of the visible light source for photopolymerization in the present invention include sunlight, halogen lamp, xenon lamp, plasma arc lamp and light emitting diode, and a light emitting diode having an emission peak at a wavelength of 380 to 420 nm is preferred. Examples of the ultraviolet light source include fluorescent tube having an emission peak at a wavelength of 379 nm and LED light emitting diode.

The artificial nail composition of the present invention can optionally contain various known additives. Examples of additives include inorganic fillers, organic and inorganic composite fillers, polymerization inhibitors, colorants, discoloration inhibitors, fluorescent agents, ultraviolet absorbers, antibacterial agents, and volatile organic solvents.

### EXAMPLES

The present invention will be described in detail by way of Examples, but the invention is not limited to the following Examples.

Abbreviations (Chemical Names) used in Examples
1. Compound having at least one radical polymerizable unsaturated double bond in the molecule
23G: tricosaethylene glycol dimethacrylate
UDMA: di(methacryloxyethyl)trimethylhexamethylenediurethane
2. Acylphosphine oxide-type photopolymerization initiator
MAPO: 2,4,6-trimethylbenzoyl-diphenyl-phosphine oxide
BAPO: bis(2,4,6-trimethylbenzoyl)-phenylphosphine oxide
3. α-hydroxyalkylphenone-type photopolymerization initiator
IRG-184: 1-hydroxy-cyclohexyl-phenyl-ketone
DAR-1173: 2-hydroxy-2-methyl-1-phenyl-propan-1-one
BAMP: 2-benzyl-2-(dimethylamino)-4′-morpholinobutyrophenone
4. Colorant
JR805: titanium oxide (manufactured by TAYCA CORPORATION)
5. Inorganic filler
R972: silica filler (manufactured by NIPPON AEROSIL CO., LTD.)
   Equipments and Apparatuses used in Tests
1. Ultraviolet photopolymerization apparatus
AKZENTZUV lamp (Model UVL-92, manufactured by TAKIGAWA CO., LTD.)
2. Visible photopolymerization light source
LED direct bar illumination (Model IDB-C50/15UV-400, peak wavelength of 405 nm: manufactured by Imac Co., Ltd.)

### Preparation of Artificial Nail Composition

Raw materials were weighed in accordance with the formulations shown in Table 1 and Table 2, mixed under atmospheric pressure at 60° C. for 3 hours and then mixed under atmospheric pressure at 23±2° C. for 12 hours to obtain high-viscosity transparent liquids. The resultant high-viscosity transparent liquids were degassed under 40 to 45 Torr.

### Evaluation 1 of Curability

As evaluation of curability, a photopolymerization exotherm temperature of the composition was measured. A cylindrical plastic container having a diameter of 6 mm was filled with 1 ml of the artificial nail composition shown in Table 1. A piercing thermocouple (manufactured by ANRITSU-METER) was inserted into the cylindrical plastic container from the upper portion and the exotherm temperature was measured at intervals of one second. As an example of ultraviolet curing, the cylindrical plastic container filled with the artificial nail composition was fixed at the center in an ultraviolet photopolymerization apparatus using an AKZENTZUV lamp. As an example of visible light curing, the cylindrical plastic container filled with the artificial nail composition was irradiated with light from the side using LED direct bar illumination. In both cases, the time at a peak of an exotherm temperature curve due to photopolymerization was recorded as a curing time, letting a light irradiation time to be zero second. At the same time, the temperature at a peak of an exotherm temperature curve due to photopolymerization was recorded as a curing temperature. For comparison with commercially available gel nails, the curing time and the curing temperature of AKZENTZ (color tone: OPTIONS CLEAR) and CALJRL (color tone: 0) were measured in the same manner. The measured curing time and curing temperature are shown in Table 1. Both AKZENTZ and CALJRL are products manufactured by TAKIGAWA CO., LTD. and AKZENTZ contains a urethane methacrylate oligomer, hydroxyethyl methacrylate, hydroxycyclohexyl phenyl ketone, benzophenone, butyl perbenzoate, and paraffin, while CALJRL contains a polyethylene methacrylate copolymer, hydroxyalkyl methacrylate, butyl perbenzoate and a photopolymerization initiator.

### Evaluation of Yellowness

Yellowness was evaluated in accordance with JIS K 7373: 2006 (Plastic—Method for Determination of Yellowness and Degree of Yellowness). A ring-type stainless steel mold having a diameter (inner diameter) of 15 mm and a thickness of 2 mm was filled with an artificial nail composition. The shape was modified and the surface to be irradiated with light was shielded from air by contacting under pressure using slide glasses from upper and lower directions. Using an ultraviolet polymerization apparatus (AKZENTZ UV LAMP), photopolymerization was performed for 3 minutes to prepare a disk-shaped cured body of the artificial nail composition. This resultant disk-shaped cured body was used as a disk-shaped specimen for measurement of yellowness, and a degree of yellowness was measured using Spectrophotometer: CM-2002 (manufactured by Konica Minolta Holdings, Inc.) For comparison with commercially available gel nails, a degree of yellowness of AKZENTZ (color tone: OPTIONS CLEAR) and CALJRL (color tone: 0) was measured in the same manner.

### [Measuring Conditions]

a) Standard number: JIS K 7373: 2006
b) Kind and shape of specimen: Plate-shaped specimens having a diameter of 15 mm and a thickness of 2 mm of cured bodies of prototype artificial nail compositions (Examples 1 to 19, Comparative Examples 1 to 7), and commercially available products (AKZENTZ (color tone: OPTIONS CLEAR) (Comparative Example 8), CALJRL (color tone: 0) (Comparative Example 9)) were used.
c) Illuminant and color system: An auxiliary illuminant C was used and an XYZ color system was used.
d) The measurement was carried out by a reflection measuring method.
e) Geometrical optical conditions defined in JIS Z 8722 used for measurement: Conditions c [8/0]
f) Model and name of test apparatus: Spectrophotometer (Model CM-2002, manufactured by Konica Minolta Holdings, Inc.)
g) Caul of back surface of specimen: Standard white surface was used.
The degree of yellowness measured (sometimes referred to as YI) is shown in Table 1.

### Evaluation 2 of Curability

Internal curability in the composition containing a colorant was evaluated. A ring-type stainless steel mold having a diameter of 15 mm and a thickness of 2 mm was filled with an artificial nail composition prepared according to the formulation shown in Table 2. The shape was modified and the surface to be irradiated with light was shielded from air by contacting under pressure using slide glasses from upper and lower directions. Using an ultraviolet polymerization apparatus (AKZENTZ UV LAMP), photopolymerization was performed for 3 minutes by LED direct bar illumination, as an example of ultraviolet curing. After 3 minutes, a curing thickness of a cured body of the irradiated surface was measured with a micrometer. The measurement results are shown in Table 2.

TABLE 1
Formulation of raw materials of artificial nail composition and results of
evaluation 1 of curability, results of evaluation of yellowness

| | | | | | | | | | LED light source | | | | |
|---|---|---|---|---|---|---|---|---|---|---|---|---|---|
| | Amount of raw materials of artificial nail | | | | | | | | (405 nm) | | UV lamp | | |
| | composition [Parts by weight] | | | | | | | Inorganic | Curing | Curing | Curing | Curing | |
| | | | | | IRG- | DAR- | | filler | time | temperature | time | temperature | |
| Examples | 23G | UDMA | MAPO | BAPO | 184 | 1173 | BAMP | (R972) | [sec] | [° C.] | [sec] | [° C.] | Yellowness |
| Example 1 | 40.00 | 60.00 | 1.00 | | | | | | 45 | 74.5 | 66 | 76.0 | 6.5 |
| Example 2 | 40.00 | 60.00 | 0.50 | | | | | | 36 | 73.2 | 36 | 86.7 | 3.3 |
| Example 3 | 40.00 | 60.00 | 0.10 | | | | | | 40 | 80.4 | 15 | 101.6 | 0.2 |
| Example 4 | 40.00 | 60.00 | 0.05 | | | | | | 51 | 80.9 | 16 | 98.0 | 0.2 |
| Example 5 | 40.00 | 60.00 | | 1.00 | | | | | 77 | 50.6 | 38 | 92.6 | 26.4 |
| Example 6 | 40.00 | 60.00 | | 0.50 | | | | | 45 | 64.3 | 31 | 87.5 | 14.0 |
| Example 7 | 40.00 | 60.00 | | 0.10 | | | | | 32 | 78.5 | 14 | 90.0 | 1.6 |
| Example 8 | 40.00 | 60.00 | | 0.05 | | | | | 39 | 88.3 | 13 | 96.7 | 0.9 |
| Example 9 | 40.00 | 60.00 | 1.00 | | | 1.00 | | | 57 | 62.2 | 16 | 65.0 | 5.2 |
| Example 10 | 40.00 | 60.00 | 0.50 | | | 0.50 | | | 47 | 70.5 | 27 | 59.8 | 4.7 |
| Example 11 | 40.00 | 60.00 | 0.10 | | | 0.10 | | | 35 | 81.4 | 19 | 97.3 | 1.0 |
| Example 12 | 40.00 | 60.00 | 0.05 | | | 0.05 | | | 43 | 83.2 | 13 | 95.1 | 0.5 |
| Example 13 | 50.00 | 50.00 | 2.00 | | | | | 1.00 | 63 | 79.5 | 65 | 78.2 | 10.8 |
| Example 14 | 50.00 | 50.00 | 4.00 | | | | | 0.50 | 75 | 74.2 | 71 | 78.0 | 11.1 |
| Example 15 | 50.00 | 50.00 | 2.00 | | | 2.00 | | 1.00 | 92 | 86.3 | 76 | 82.3 | 11.5 |
| Example 16 | 50.00 | 50.00 | 0.25 | 0.10 | | 0.25 | | | 38 | 105.2 | 34 | 103.9 | 6.1 |
| Example 17 | 50.00 | 50.00 | 0.50 | 0.10 | | 0.50 | | | 53 | 102.9 | 47 | 95.6 | 6.5 |
| Example 18 | 50.00 | 50.00 | 1.00 | 0.10 | | 1.00 | | | 93 | 93.8 | 66 | 96.7 | 9.6 |
| Example 19 | 50.00 | 50.00 | 2.00 | 0.10 | | 2.00 | | | 98 | 82.1 | 86 | 78.7 | 10.6 |

| | | | | | | | |
|---|---|---|---|---|---|---|---|
| Comparative | 40.00 | 60.00 | 3.00 | (Note) 1 | 14 | 88.8 | 1.1 |
| Example 1 | | | | | | | |
| Comparative | 40.00 | 60.00 | 1.00 | (Note) 2 | 14 | 98.2 | 0.4 |
| Example 2 | | | | | | | |
| Comparative | 40.00 | 60.00 | 0.10 | (Note) 3 | 31 | 88.6 | 0.1 |
| Example 3 | | | | | | | |

| | | | | | | | | |
|---|---|---|---|---|---|---|---|---|
| Comparative | 40.00 | 60.00 | 3.00 | 36 | 70.3 | 30 | 76.2 | 58.7 |
| Example 4 | | | | | | | | |
| Comparative | 40.00 | 60.00 | 1.00 | 34 | 78.1 | 28 | 80.3 | 52.6 |
| Example 5 | | | | | | | | |
| Comparative | 40.00 | 60.00 | 0.50 | 52 | 83.7 | 14 | 92.3 | 52.4 |
| Example 6 | | | | | | | | |
| Comparative | 40.00 | 60.00 | 0.05 | 69 | 77.2 | 13 | 95.2 | 31.5 |
| Example 7 | | | | | | | | |

| | | | | | |
|---|---|---|---|---|---|
| Comparative | AKZENTZ | (Note) 4 | 53 | 56.9 | 5.0 |
| Example 8 | | | | | |
| Comparative | CALJRL | (Note) 5 | 69 | 71.8 | 20.5 |
| Example 9 | | | | | |

(Note) 1-5: It was impossible to measure since neither heat generation nor curing occur after 3 minutes.

TABLE 2
Formulation of raw materials of artificial nail composition and results of
evaluation 2 of curability

| | | | | | | | | Curing thickness [μm] | |
|---|---|---|---|---|---|---|---|---|---|
| | | | | | | | | LED light | |
| | Amount of raw materials of artificial nail composition [Parts by weight] | | | | | | | source | |
| Examples | 23G | UDMA | JR-805 | MAPO | BAPO | IRG-184 | DAR-1173 | (405 nm) | UV lamp |
| Example 20 | 38.46 | 57.69 | 2.88 | 0.96 | | | | 680 | 156 |
| Example 21 | 37.38 | 56.07 | 5.61 | 0.93 | | | | 404 | 83 |
| Example 22 | 38.46 | 57.69 | 2.88 | | 0.96 | | | 405 | 394 |
| Example 23 | 37.38 | 56.07 | 5.61 | | 0.93 | | | 269 | 276 |
| Example 24 | 38.10 | 57.14 | 2.86 | 0.95 | | | 0.95 | 669 | 213 |
| Example 25 | 37.04 | 55.56 | 5.56 | 0.93 | | | 0.93 | 443 | 131 |
| Comparative | 38.46 | 57.69 | 2.88 | | | 0.96 | | 0 | 40 |
| Example 10 | | | | | | | | | |
| Comparative | 37.38 | 56.07 | 5.61 | | | 0.93 | | 0 | 13 |
| Example 11 | | | | | | | | | |

1. An artificial nail composition comprising:
a component (a): a compound having at least one radical polymerizable unsaturated double bond in the molecule, and
a component (b): a photopolymerization initiator selected from (i) an acylphosphine oxide-type photopolymerization initiator and (ii) a mixture of an acylphosphine oxide-type photopolymerization initiator and an α-hydroxyalkylphenone-type photopolymerization initiator, wherein the amount of the component (b) is 0.05 to 4.00 parts by weight with respect to 100 parts by weight of the component (a).

2. The artificial nail composition according to claim 1, wherein (b) the acylphosphine oxide-type photopolymerization initiator is monoacylphosphine oxide and/or bisacylphosphine oxide and the α-hydroxyalkylphenone-type photopolymerization initiator is 1-hydroxy-cyclohexyl-phenyl-ketone or 2-hydroxy-2-methyl-1-phenyl-propan-1-one.

3. The artificial nail composition according to claim 1, wherein a visible or ultraviolet light source is used as a photopolymerization light source upon curing.

4. The artificial nail composition according to claim 2, wherein a visible or ultraviolet light source is used as a photopolymerization light source upon curing.
